(19) United States
(12) Patent Application Publication
(10) Pub. No.: US 2014/0170649 A1
(43) Pub. Date: Jun. 19, 2014

(54) LATERAL FLOW DETECTION OF TARGET SEQUENCES

(71) Applicants: Joseph F. Krebs, Austin, TX (US); Paul Morrison, Austin, TX (US); Marianna Goldrick, Austin, TX (US)

(72) Inventors: Joseph F. Krebs, Austin, TX (US); Paul Morrison, Austin, TX (US); Marianna Goldrick, Austin, TX (US)

(73) Assignee: BIOO SCIENTIFIC CORPORATION, Austin, TX (US)

(21) Appl. No.: 14/039,908

(22) Filed: Sep. 27, 2013

Related U.S. Application Data
(60) Provisional application No. 61/706,444, filed on Sep. 27, 2012.

(51) Int. Cl. C12Q 1/68

(52) U.S. Cl. CPC C12Q 1/6804
USPC 435/6.11

(57) ABSTRACT

Provided is a sensitive method to specifically detect nucleic acid sequences using a combination of DNA replication based signal amplification (e.g., PCR) and lateral flow analyte detection. The disclosed method uses a dual-labeled DNA probe complementary to a region of the target DNA sequence. Cleavage of the probe, caused by the presence of the amplified DNA target, is detected using lateral flow methods.

### PRIORITY CLAIM

This application claims the benefit of U.S. Provisional Application No. 61/706,444 filed on Sep. 27, 2012.

### BACKGROUND OF THE INVENTION

1. Field of the Invention

The present invention generally relates to the detection of nucleic acid sequences in food, feed and other biological samples.

2. Description of the Relevant Art

Nucleic acids sequences are often highly specific and can be used for diagnostic purposes to detect a wide array of biomarkers, genetic diseases and pathogens such as bacteria, viruses, fungi and parasites. Detection of specific nucleic acid sequences can be performed in a wide variety of sample types including biological tissues and fluids as well as other samples such as food, feed and environmental samples.

PCR is a particularly useful method to detect DNA sequences because the method greatly amplifies the concentration of the target sequence during the reaction, thereby increasing detection sensitivity. In these methods, sequence-specific forward and reverse oligonucleotides direct the exponential replication of the target DNA sequence. In particular quantitative PCR (qPCR) techniques are well suited for sensitive fluorescence-based detection of DNA sequences.

Real-time PCR methods rely on the direct evolution of fluorescence signal. Real-time PCR methods are performed using a specialized instrument which is able to cycle the reaction temperature to perform the polymerase chain reaction cycles as well as to measure the increase in fluorescence of the samples caused by the production of the PCR product. While these instruments permit very sensitive sequence detection, they have several limitations which limit their ability to be used in outside of the specialized laboratory environment and by analysts with limited resources: they are delicate, expensive and require regular periodic maintenance to maintain functionality. It would therefore be highly desirable to develop a method to detect amplified DNA sequences which did not require expensive instrumentation using fluorescence signals.

### SUMMARY OF THE INVENTION

A method for detecting the presence a target DNA sequence in a sample includes amplifying the target DNA sequence by applying polymerase chain reaction (PCR), to the sample, wherein the PCR is performed using a composition. The composition includes: a thermostable DNA polymerase having 5′ exonuclease activity; forward and reverse amplification primers; deoxyribonucleotides; and a probe oligonucleotide that is complementary to an internal region of the target DNA sequence, wherein the probe oligonucleotide comprises a 5′ modification, the 5′ modification comprising a first moiety capable of associating with a first affinity reagent, and wherein the probe oligonucleotide comprises a 3′ modification, the 3′ modification comprising a second moiety capable of associating with a second affinity reagent. A portion of the PCR composition after amplification of the target DNA sequence is mixed with a mobile reporter agent to form a mobile reporter-PCR composition mixture, wherein said mobile reporter agent comprises the second affinity reagent, and wherein the second affinity reagent binds to the second moiety of the probe oligonucleotide. The mobile reporter-PCR composition mixture is introduced into a lateral flow strip under conditions that allow the probe oligonucleotide associated with the mobile reporter agent to migrate along the strip. The probe oligonucleotide associated with the mobile reporter agent is captured by first affinity reagents positioned at a detection position of the lateral flow strip. The first affinity reagent binds to the first moiety on the probe oligonucleotide to inhibit further lateral flow of the probe oligonucleotide associated with the mobile reporter agent. The detection position of the lateral flow strip is analyzed to determine the intensity of the mobile reporter agent captured at the detection position, wherein the intensity of the mobile reporter agent indicates the presence of the target DNA in the sample.

In some embodiments, the second affinity reagent is a second antibody, and the second moiety is an antigen of the second antibody. In some embodiments, the first affinity reagent at the detection position of the lateral flow strip is an antibody capable of binding to the first moiety of the probe oligonucleotide, wherein the first moiety is an antigen of the first affinity reagent. Both the first and second affinity reagents may be antibodies. In one embodiment, the second affinity reagent on the mobile reporter agent is streptavidin and the second moiety on the probe oligonucleotide is biotin.

In one embodiment, the mobile reporter agent is a fluorescent compound. In another embodiment, the mobile reporter agent is a colloidal gold particle. The lateral flow strip may be composed of nitrocellulose. The thermostable DNA polymerase may be natural or recombinant Taq from Thermus aquaticus.

### BRIEF DESCRIPTION OF THE DRAWINGS

Advantages of the present invention will become apparent to those skilled in the art with the benefit of the following detailed description of embodiments and upon reference to the accompanying drawings in which:

FIGS. 1A and 1B depict schematic diagrams of a target DNA sequence directed cleavage of complementary dual-labeled probes using DNA polymerase;

FIGS. 2A and 2B depict schematic diagrams of the detection of target DNA-dependent cleavage of dual labeled probe using a lateral flow device; and

FIGS. 3A and 3B show schematic diagrams of the detection of target DNA sequences using the Polymerase-assisted Decoupling (PAD) method.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

The method described in this disclosure can detect DNA sequences (such as pathogen DNA) without the use of optical fluorescence techniques. The present method uses a combination of PCR (using a DNA polymerase possessing 5′ exonuclease activity) and lateral flow detection to amplify and detect, respectively, specific DNA sequences in a variety of sample types. The use of a novel method to detect specific DNA polymerization, known as Polymerase-assisted Decoupling (PAD), using a new type of lateral flow detection, eliminates the need to detect samples using costly fluorescence detection instruments.

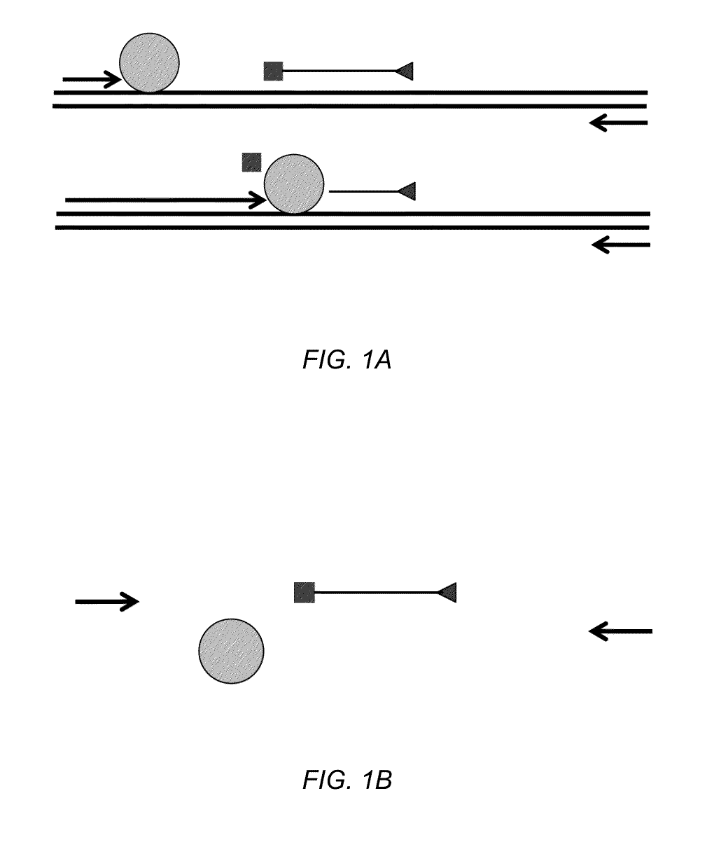

In this method, the target DNA sequence is amplified using the polymerase chain reaction using pairs of flanking primers complementary to the target sequence, deoxynuxcleotides, buffer and DNA polymerase (possessing 5′ to 3′ exonuclease activity). The PCR reaction also contains dual-labeled probes (uniquely labeled at both the 5′ and 3′ ends with affinity capture groups) complementary to internal regions of the amplicon sequence. As the DNA target sequence is amplified during the PCR reaction, the dual labeled probe specifically hybridizes to an internal sequence within the PCR product. When the dual labeled probe is hybridized to the new synthesized PCR product, it can be cleaved by the 5′ exonuclease activity of the DNA polymerase as it is synthesizing the complementary DNA strand (extending the forward primer annealed to the 5′ flanking region of the target DNA sequence). In this scheme, cleavage of the probe requires hybridization of the probe to the amplified DNA. If the target DNA is not present in the reaction, no amplified target sequence is produced, so cleavage of the probe cannot occur; probe cleavage requires the presence of the target sequence. FIG. 1 depicts target DNA sequence directed cleavage of complementary dual-labeled probes using DNA polymerase. In FIG. 1A The polymerase (circle) is shown as synthesizing a complementary DNA strand directed by the forward primer. When the polymerase reaches the probe, it will degrade the downstream oligonucleotide probe hybridized to the DNA template. If the probe is labeled with unique labels on its ends (square and triangle) the degradation will disconnect the labels. FIG. 1B shows that if no target sequence is present in the reaction, specific DNA synthesis will not occur and the probe will remain intact.

In this manner the method can be used to detect target nucleic acid sequences in samples. A signal from one of the labeled ends will be present at the immobilized zone in the lateral flow device if no target is present. A reduction in signal at the zone will be observed if the target is present in the sample. Therefore this method can be used to conveniently detect the presence of a target DNA sequence in samples.

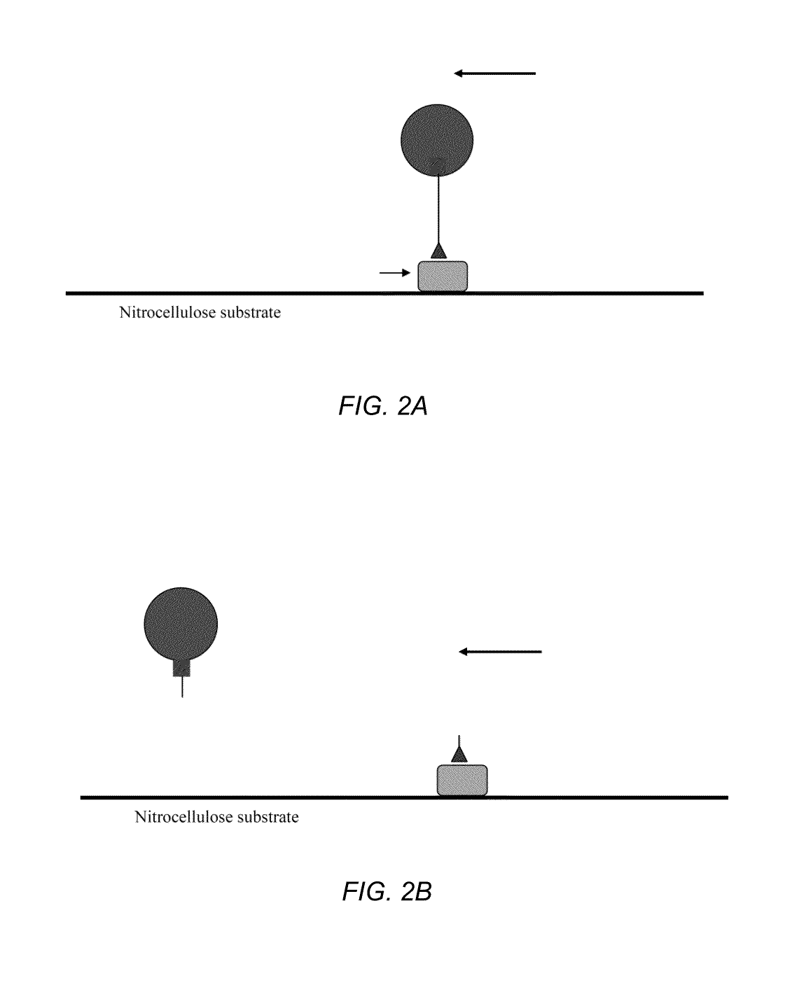

Since the probe molecule contains 2 distinct labels, its integrity and/or degradation can be detected by using the probes as linkers to attach an indicator (e.g., a fluorescent compound or coated colloidal gold particles) to immobilized protein zones on nitrocellulose on a lateral flow device. FIG. 2 depicts the detection of target DNA-dependent cleavage of dual labeled probe using a lateral flow device. FIG. 2A depicts the scenario when no target sequence is present. If no target sequence is present in the PCR reaction, the probe remains intact. When the probe is mixed with an indicator (circle) which binds one of its labeling groups (square) and the mixture is allowed to flow across a lateral flow device, the other label on the probe (triangle) will bind to an immobilized capture reagent zone (rectangle) on the device which will cause the visible signal from the indicator to accumulate at the zone. FIG. 2B shows that if the target DNA sequence causes degradation of the probe during the PCR reaction, the portion of the probe bound to the indicator is no longer able to bind to the an immobilized capture reagent zone. Thus, no significant signal forms at the capture zone line when the target DNA sequence is present.

The amplification of specific target DNA sequences can thus be detected by the visualization of indicators attached to probes that bind to a capture line on a lateral flow device.

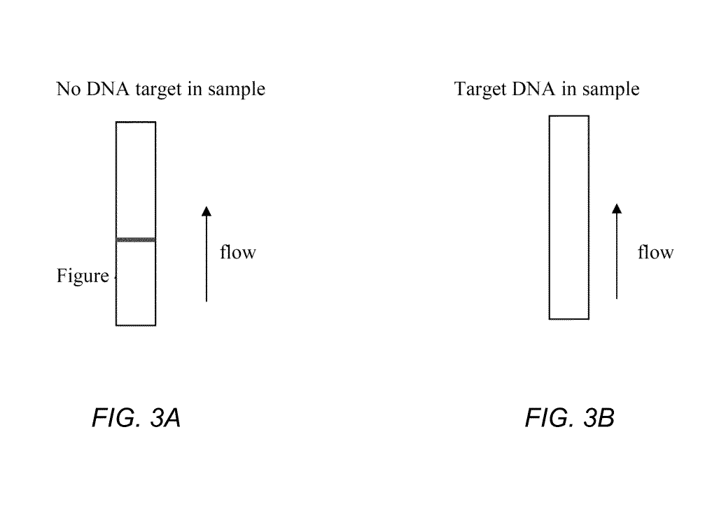

FIG. 3 shows the detection of target DNA sequences using the Polymerase-assisted Decoupling (PAD) method. The left side (FIG. 3A), shows that the target DNA is not present in the sample—the probe remains intact and is able to link the indicator to the capture zone on the lateral flow device. This causes a visible signal at the zone. On the right side (FIG. 3B), the target DNA is present in the sample—the probe is cleaved and unable to bind the indicator to the zone. This causes a decrease in the signal at the zone.

Target DNA sequences can be amplified in PCR reactions using pairs of complementary flanking primers, deoxynuxcleotides, buffer and DNA polymerase. The PCR method can also be used to detect RNA sequences if the RNA is first converted to DNA using the reverse transcriptase enzyme. Target sequence amplification can be used to detect DNA sequences in a variety of samples for a range of uses including pathogen detection, forensic identification, and gene expression analysis. In the present disclosure, the amplification of DNA by polymerase can also be monitored if these reactions also contain probes (uniquely labeled at both the 5′ and 3′ ends) complementary to internal regions of the target DNA sequence as well as a DNA polymerase possessing 5′ to 3′ exonuclease activity. Such probes are degraded in a template-dependent manner when they hybridize to the target DNA and are degraded by the DNA polymerase as it extends the forward primer. Since these probes contain 2 distinct labels, their integrity and/or degradation can be detected (after completion of the PCR reaction) by using them as linking agents to attach detectable molecules (e.g. green fluorescent protein) or detectable particles (e.g. coated colloidal gold particles) to an immobilized protein zone on nitrocellulose on a lateral flow device. Therefore the amplification of specific target DNA sequences can be detected by the reduction of visible colloidal gold particles bound to a capture line (known as the test line) on a lateral flow device.

If these probes contain 2 distinct labels, their degradation associated with target sequence amplification (via polymerase 5′ exonuclease activity) prevents attachment of the colloidal gold particle to the immobilized protein on the nitrocellulose membrane. The appropriate polymerase will degrade the labeled probe hybridized to the target DNA sequence as it is extends the primer across the target sequence (amplicon). This template-dependent cleavage will destroy the ability of the probe to attach the colloidal gold to the immobilized protein zone on the nitrocellulose when the reaction components are allowed to flow from one end of the nitrocellulose to the other (through the immobilized protein zone known as the test line).

Therefore the presence or absence of target DNA sequence in a sample can be determined by amplifying a sequence of target DNA (by PCR) in the presence of a single-stranded complementary probe labeled at each end with a different affinity capture group (such as biotin or an antibody binding epitope). If one label can bind a capture reagent immobilized on the lateral flow device and the other label is capable of binding a detectable particle such as a colloidal gold particle, latex bead or quantum dot, the accumulation of color or fluorescence can be used to monitor the binding and flow of the reaction components through the nitrocellulose.

The method has many important applications as a diagnostic technique. The method can be used to detect pathogens derived from a variety of sources including tissues, biological fluids, environmental samples, food and feed. For example the method can be used to detect pathogenic E. coli bacteria in food samples after the samples have been cultured in appropriate growth media to facilitate detection. In another embodiment, the method can be used to detect pathogens (bacterial, viral or other) in biological or clinical samples.

While the present description and attached examples demonstrate the utility of the invention to detect DNA sequences, it will be readily apparent to a practitioner skilled in the art that the method can be used to detect target RNA sequences if reverse transcriptase is first added to the reaction to produce cDNA molecules complementary to the target RNA sequences in the sample. In such instances, the method can be used to detect RNA sequences since target RNA molecules (via cDNA) would cause a signal decrease on the immobilized zone within the lateral flow device.

Furthermore, while the present description and examples describe detection of target nucleic acid sequences using PCR reactions, it will be readily apparent to a practitioner that the method can also be used to detect DNA sequences amplified using an isothermal DNA amplification method provided the probe is able to hybridize to the target sequence and the DNA polymerase possesses a significant 5′ exonuclease activity which is comparable to that of the DNA polymerase from Thermophilus aquaticus and able to efficiently degrade complementary internal probes hybridized to the amplicon to reduce the amount of bound colloidal gold bound to the immobilized zone.

To confirm that the lateral flow test was performed properly and to normalize for fluctuations in signal intensities unrelated to gene expression variations, an additional capture zone (known as the control line) can be added to the lateral flow device. This zone generally contains an affinity capture reagent (unrelated to the analyte-specific capture reagent at the test line) capable of binding colloidal gold (or other detectable) particles which flow past the test line.

In another embodiment, the method can be used to simultaneously detect the presence of multiple target sequences. In this embodiment, the PCR reactions contain multiple sets of complementary amplification primers and dual-labeled probes. The probe for each target sequence contains unique paired combinations of affinity capture groups to permit unique coupling of detection particles to target specific zones of immobilized capture reagent on the lateral flow device. Affinity capture groups for the DNA probe would include but not be limited to fluorescein, biotin, TAMRA, digoxigenin, dansyl, and Texas Red. These affinity capture groups are especially useful for multiplex applications because they are commonly used to label the ends of DNA oligonucleotides, and they can be specifically immobilized by commercially-available antibody reagents. In this instance the PCR reaction would be used to specifically degrade the probes if its target DNA sequence is present in the reaction. After completion of the PCR, the reaction solution would be mixed with a mixture of colloidal gold particles; each particle is coated with a specific reagent to capture one of the probes (capture groups) in reaction mix. After the probes and detection particles have complexed, the mixture may be passed through a lateral flow device containing detection zones; each zone contains unique affinity capture reagent specific for one of the capture groups. A reduced visible signal will be observed for each target sequence present in the test sample. In this manner, the method can be used to detect the presence of multiple target sequences from a single PCR reaction and lateral flow test. If desired, this multiplex method can include detection of a positive control target sequence which is certain to be present in all PCR reactions. This positive control can be used to help confirm successful degradation of the probe during the PCR reaction.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Detection of Eae synthetic target

The disclosed methods and compositions were used to detect a synthetic target derived from the E. coli eae gene. The synthetic target was made by annealing two partially complementary oligonucleotides that spanned the Eae target and included about 20 additional bases on each side in a buffer containing 100 mM NaCl, then carrying out 20 cycles of PCR using Taq polymerase to extend the overlaps to make the double-stranded target region, then using 5 μL of the amplicon as template and carrying out 30 additional cycles of PCR using primers annealing to the ˜20 bases at the each end of the amplicon to produce a large amount of the synthetic target. The product was checked on a 2% agarose gel to confirm the expected size of 140 bp; no non-specific side products were observed. The synthetic target was then diluted by 103 and 106 in 10 mM Tris pH 8/0.1 mM EDTA, and used as template in a series of 50 μL PCRs to produce amplicons of ˜101 bp. Negative control reactions contained water instead of diluted synthetic template. The sequences of the Forward and Reverse primers and internal dual labeled hybridization probe used in the PCRs and resulting Eae amplicons are described in USDA publication “Primer and Probe Sequences and Reagent Concentrations for non-O157 STEC Real Time PCR Assay”. The concentration of probe used differed from that described in the USDA assay; the final probe concentration disclosed in that document is 200 nanomolar (nM), whereas the probe concentrations used in the reaction of this example were 100 nM and 30 nM. The oligonucleotide probe included a 5′ FAM modification and a 3′-Biotin. These modifications were used for the capture and reporting steps of the disclosed assay. The amplification was carried out for 35 cycles, where each cycle consisted of denaturation for 10 sec at 95° C., followed by annealing/extension for 1 min at 59° C. The 35 cycles were followed by a final extension at 72° C. for 3 min.

Following PCR amplification of the Eae template, 1 μL of PCR mixture is added to and briefly mixed with 80 μL of a PBS solution containing 0.5% Tween-20 and 40 nm gold nanoparticles coated with streptavidin. Any intact dual-labeled probe remaining in the PCR mixture binds to the streptavidin coated gold particles via the 3′ biotin label. A 5 mm wide nitrocellulose-backed strip, with an anti-FAM capture antibody immobilized at the test line area and an anti-streptavidin antibody immobilized at the control line area, is then immersed into the liquid PCR reaction and gold particle mixture for 5 minutes. The antibodies are deposited and dried as horizontal lines at the test line area and the control line area using an XYZ spraying system from BioDot Inc. The liquid mixture will flow by capillary action up the nitrocellulose membrane on the strip, first across the test line area followed by the control line area. Gold particles linked to intact dual-labeled probe are captured by the immobilized anti-FAM antibody at the test line area, creating a bright red signal proportional to the concentration of intact dual-labeled probe remaining in the PCR reaction mixture. The dual-labeled probe acts as a tether between the gold particles and the immobilized anti-FAM antibody at the test line. Degraded dual-labeled probe either binds to the streptavidin coated gold particles or the immobilized anti-FAM antibody, but not both, and thus will not form a red signal at the test line. The remaining gold particles are captured by the anti-streptavidin antibody at the control line. The presence of EAE template in the PCR mixture is determined by the decreased visual signal at the test line (FIG. 4). Interpretation of the intensity of the signal on the strip is performed visually or using an optical strip reader from Detekt Biomedical (Austin, Tex.).

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for detecting the presence a target DNA sequence in a sample comprising:
amplifying the target DNA sequence by applying polymerase chain reaction (PCR), to the sample, wherein the PCR is performed using a composition comprising:
a thermostable DNA polymerase having 5′ exonuclease activity;
forward and reverse amplification primers;
deoxyribonucleotides; and
a probe oligonucleotide that is complementary to an internal region of the target DNA sequence, wherein the probe oligonucleotide comprises a 5′ modification, the 5′ modification comprising a first moiety capable of associating with a first affinity reagent, and wherein the probe oligonucleotide comprises a 3′ modification, the 3′ modification comprising a second moiety capable of associating with a second affinity reagent;
mixing a portion of the PCR composition after amplification of the target DNA sequence with a mobile reporter agent to form a mobile reporter-PCR composition mixture, wherein said mobile reporter agent comprises the second affinity reagent, wherein the second affinity reagent binds to the second moiety of the probe oligonucleotide;
introducing the mobile reporter-PCR composition mixture into a lateral flow strip under conditions that allow the probe oligonucleotide associated with the mobile reporter agent to migrate along the strip, wherein the probe oligonucleotide associated with the mobile reporter agent is captured by first affinity reagents positioned at a detection position of the lateral flow strip, and wherein the first affinity reagent binds to the first moiety on the probe oligonucleotide to inhibit further lateral flow of the probe oligonucleotide associated with the mobile reporter agent;
analyzing the detection position of the lateral flow strip to determine the intensity of the mobile reporter agent captured at the detection position, wherein the intensity of the mobile reporter agent indicates the presence of the target DNA in the sample.

2. The method of claim 1, wherein the second affinity reagent is a second antibody, and the second moiety is an antigen of the second antibody.

3. The method of claim 1, wherein the first affinity reagent at the detection position of the lateral flow strip is an antibody capable of binding to the first moiety of the probe oligonucleotide, wherein the first moiety is an antigen of the first affinity reagent.

4. The method of claim 1, wherein the second affinity reagent on the mobile reporter agent is streptavidin and the second moiety on the probe oligonucleotide is biotin.

5. The method of claim 1, where the mobile reporter agent is a fluorescent compound.

6. The method of claim 1, where the mobile reporter agent is a colloidal gold particle.

7. The method of claim 1, wherein the lateral flow strip comprises of nitrocellulose.

8. The method of claim 1, wherein the thermostable DNA polymerase is natural or recombinant Taq from Thermus aquaticus.
